(19) United States
(12) Patent Application Publication
(10) Pub. No.: US 2022/0117870 A1
(43) Pub. Date: Apr. 21, 2022

(54) COMPOSITION AND METHODS FOR IMPARTING A LIKING, CARING OR FRESH ATTRIBUTE

(71) Applicant: International Flavors & Fragrances Inc., New York, NY (US)

(72) Inventors: Celine BAREL, Union Beach, NJ (US); Anne-Sophie CARIOU, Union Beach, NJ (US); John P. CHERKAUSKAS, Jr., Union Beach, NJ (US); Peter COATES, Union Beach, NJ (US); Lorraine Miche DE MALLERAY, Union Beach, NJ (US); Loc DONG, Union Beach, NJ (US); Gregoire HAUSSON, Union Beach, NJ (US); Judith KERSCHNER, Union Beach, NJ (US); Celine MANETTA, Union Beach, NJ (US); Arnaud MONTET, Union Beach, NJ (US); Khaled NASROUNE, Union Beach, NJ (US); Camille POUY, Union Beach, NJ (US); Franc T. SCHIET, Union Beach, NJ (US)

(21) Appl. No.: 17/286,089

(22) PCT Filed: Oct. 16, 2018
(86) PCT No.: PCT/US2018/056014

§ 371 Date: Apr. 16, 2021

(51) Int. Cl. A61K 8/49; A61K 8/34; A61K 8/35; A61K 8/37; A61Q 13/00

(52) U.S. Cl. CPC A61K 8/4973; A61K 8/345; A61K 2800/591; A61K 8/37; A61Q 13/00; A61K 8/35

(57) ABSTRACT

Fragrance compositions and methods for imparting to consumers fresh, caring and liking attributes are provided which include Amber Xtreme™ in combination with one or more synergistic fragrance ingredients including citronellol, damascenone, geraniol, helional, muscemor or ambrettolide.

### BACKGROUND

Ingredient contribution to a formulation is a key success factor for fragrance creation. Conventionally, perfumers use their experience and know how to create new fragrance combinations. However, an analysis of what drives benefit attributes has not been described.

Amber Xtreme™ is a woody/amber fragrance ingredient disclosed U.S. Pat. Nos. 6,632,788 and 7,160,852. Since its discovery, Amber Xtreme™ has been described for use in complex fragrance accords containing additional ingredients such as geraniol (WO 2018/119469 A1, WO 2018/119466 A1, WO 2018/119471 A1, WO 2018/005844 A1, WO 2018/005839 A1, WO 2017/214446 A1, and WO 2017/214455 A1) and helional (U.S. Pat. No. 9,714,401 B2, U.S. Pat. No. 9,814,661 B2, WO 2017/070132 A1, WO 2017/070131 A1, WO 2016/049398 A1, WO 2016/049407 A1, WO 2016/049458 A1, US 2017/0275564 A1, US 2017/0255725 A1, US 2017/0137753 A1, US 2017/0119917 A1, US 2017/0107462 A1, US 2017/0249408 A1, US 2017/0121633 A1, US 2016/0296656 A1, US 2016/0089464 A1, US 2016/0089462 A1, US 2016/0090555 A1, US 2016/0090556 A1, US 2016/0090557 A1, US 2016/0090558 A1, US 2016/0089317 A1, US 2016/0089318 A1, US 2016/0362631 A1, US 2016/0362630 A1, US 2016/0362629 A1, US 2016/0115424 A1).

### SUMMARY OF THE INVENTION

This invention relates to a fragrance composition and method for imparting a fresh attribute by providing a fragrance composition including about 0.07 to 1.3 wt % Amber Xtreme™, and at least one of about 0.4 to 2.0 wt % citronellol, or about 0.06 to 0.13 wt % damascenone. In some embodiments, the composition and method further include the use of one or more of about 0.9 to 14.8 wt % ambrettolide, about 0.1 to 1.9 wt % dipropylene glycol, about 14.8 to 46.7 wt % 1-(1,2,3,5,6,7,8,8a-octahydro-2,3,8,8-tetramethyl-2-naphthyl)ethan-1-one, or about 21.8 to 24.7 wt % kharismal. A consumer product containing the fragrance composition is also provided.

This invention also relates to a fragrance composition and method for imparting a caring attribute by providing about 0.6 to 5.9 wt % Amber Xtreme™, and at least one of about 0.2 to 2.0 wt % geraniol, or about 2.5 to 4.9 wt % helional. In some embodiments, the composition and method further include the use of one or more of about 0.9 to 14.8 wt % ambrettolide, about 0.1 to 1.9 wt % dipropylene glycol, about 14.8 to 46.7 wt % 1-(1,2,3,5,6,7,8,8a-octahydro-2,3,8,8-tetramethyl-2-naphthyl)ethan-1-one or about 21.8 to 24.7 wt % kharismal. A consumer product containing the fragrance composition is also provided.

This invention also relates to a fragrance composition and method for imparting a liking attribute by providing about 0.05 to 2.0 wt % Amber Xtreme™, and at least one of about 0.1 to 0.6 wt % geraniol, about 1.3 to 3.0 wt % helional, about 1.8 to 13.8 wt % muscemor, or about 0.9 to 19.0 wt % ambrettolide. A consumer product containing the fragrance composition is also provided.

### DETAILED DESCRIPTION OF THE INVENTION

A link between consumer perception and synergistic combinations of fragrances including Amber Xtreme™ has now been identified. Compared to classical approaches, the present invention provides a model for synergistic fragrance interactions and specific ranges of usages of the fragrance ingredients. The instant holistic scent design approach is multidimensional as it takes consideration who the consumer is as well as the combination of fragrance ingredients, the amount of fragrance ingredients and intended end-use applications to deliver consumer benefits. Female consumers from four regions of the world, namely North America, South America, Europe and Asia, evaluated 90 different fragrance compositions for various attributes including caring, liking and fresh attributes. This research indicated that the fragrance ingredients were analogous to puzzle pieces; each ingredient fit or did not with another ingredient. When an ingredient fit with another ingredient, there was a positive synergistic response. When an ingredient did not fit to another ingredient, there is no synergy or neutral synergy. More specifically, positive synergies between Amber Xtreme™ and at least one of citronellol, damascenone, geraniol, helional, muscemor or ambrettolide, each at particular doses, were identified in providing caring, liking and fresh attributes to a fragrance composition.

Accordingly, the present invention provides fragrance compositions and methods for imparting a fresh, caring or liking attribute by providing Amber Xtreme™ (decahydro-2,2,6,6,7,8,8-heptamethyl indenofuran; CAS No. 476332-65-7) in combination with at least one synergistic fragrance ingredient selected from the group of citronellol (3,7-dimethyloct-6-en-1ol; CAS No. 106-22-9), damascenone ((E)-1-(2,6,6-trimethyl-1-cyclohexa-1,3-dienyl)but-2-en-1-one; CAS No. 23696-85-7), geraniol (3,7-dimethyl-2,6-octadien-1-ol; CAS No. 106-24-1), helional (3-(3,4-methylendioxyphenyl)-2-methylpropanal; CAS No. 1205-17-0), muscemor ((5E)-3-methylcyclopentadec-5-en-1-one; CAS No. 82356-51-2) or ambrettolide ((9Z)-17-oxacycloheptadec-9-en-1-one; CAS No. 28645-51-4).

For the purposes of the present invention, the terms “fragrance composition,” “fragrance formulation,” and “perfume composition” mean the same and refer to a composition that is a mixture of fragrance ingredients including, for example, alcohols, aldehydes, ketones, esters, ethers, lactones, nitriles, natural oils, synthetic oils, and mercaptans, which are admixed so that the combined odors of the individual ingredients produce a pleasant or desired fragrance. Ideally, the fragrance composition of the present invention is a consumer composition including Amber Xtreme™ and one or more synergistic fragrance ingredients. The fragrance composition of the present invention may also include Amber Xtreme™, one or more synergistic fragrance ingredients, and a complementary fragrance ingredient as described herein.

A “synergistic fragrance ingredient” refers to a fragrance ingredient, which, when combined with Amber Xtreme™, imparts an attribute of liking, caring or fresh to a fragrance composition, independent of the other fragrance ingredients included in the fragrance composition. A fragrance composition “imparts” a recited attribute when a consumer smelling the fragrance composition has a primary sensation or overall impression of the recited attribute, independent of other secondary or tertiary attributes the fragrance composition may convey. For the purposes of this invention, the attribute or perception of “liking” refers the degree to which a consumer appreciates or likes a fragrance composition. Ideally, “liking” refers to a consumer “liking” or “very much liking” a fragrance, as compared to only “slightly liking,” “neither liking nor disliking,” “disliking slightly,” “disliking” or “disliking very much” the fragrance. In particular, out of about 100 consumers surveyed, at least 40%, 45%, 50%, 60%, 70% or 80% of the consumers perceived a “liking” or “very much liking” attribute to a fragrance. Further, as used herein, the attribute or perception of “caring” refers to a consumer's impression that the fragrance composition provides a feeling of protection, conveys a feeling of harmlessness and being good for one's health, suggests nourishment or moisturizing, and/or imparts a feeling of indulgence (e.g., taking care of oneself and helping one to unwind). In particular, out of about 100 consumers surveyed, at least 40%, 45%, 50%, 60%, 70% or 80% of the consumers perceived a “caring” attribute to a fragrance. Moreover, in accordance with this invention, the attribute or perception of “fresh” refers to consumer's impression that the fragrance composition gives a feeling of freshness and renewal, conveys self-confidence (e.g., has a reassuring fresh smell), and/or provides a re-energizing benefit. In particular, out of about 100 consumers surveyed, at least 40%, 45%, 50%, 60%, 70% or 80% of the consumers perceived a “fresh” attribute to a fragrance.

In accordance with the present invention, the synergistic fragrance ingredient of the fragrance composition is selected from the group of citronellol, damascenone, geraniol, helional, muscemor and ambrettolide. More specifically, synergistic fragrance ingredients and their respective amounts, which impart a fresh, caring or liking attribute when used in combination with Amber Xtreme™ are provided in Table 1.

TABLE 1

| | Wt % Amber | Wt % Synergistic Fragrance |
|---|---|---|
| Attribute | Xtreme ™ | Ingredient |
| Fresh | 0.07-1.3 wt % | 0.4-2.0 wt % Citronellol, and/or |
| | | 0.06-0.13 wt % Damascenone |
| Caring | 0.6-5.9 wt % | 0.2-2.0 wt % Geraniol, and/or |
| | | 2.5-4.9 wt % Helional |
| Liking | 0.05-2.0 wt % | 0.1-0.6 wt % Geraniol, and/or |
| | | 1.3-3.0 wt % Helional, and/or |
| | | 1.8-13.8 wt % Muscemor, and/or |
| | | 0.9-19.0 wt % Ambrettolide |

Ideally, the fragrance composition of invention imparts a fresh, caring, or liking attribute to consumers in multiple regions or countries of the world. For example, it was found that a fragrance composition containing about 0.05 to 2.0 wt % Amber Xtreme™ in combination with about 0.1 to 0.6 wt % geraniol, about 1.3 to 3.0 wt % helional, about 1.8 to 13.8 wt % muscemor, and/or about 0.9 to 19.0 wt % ambrettolide was overwhelmingly liked by consumers in North America, South America, Europe and Asia. Accordingly, in certain embodiments, a fragrance composition or consumer product containing about 0.05 to 2.0 wt % Amber Xtreme™ in combination with about 0.1 to 0.6 wt % geraniol, about 1.3 to 3.0 wt % helional, about 1.8 to 13.8 wt % muscemor, and/or about 0.9 to 19.0 wt % ambrettolide is formulated for a consumer located in any region of the world as it is recognized world-wide as imparting a liking attribute to a fragrance composition.

In another embodiment, the fragrance composition of invention imparts a fresh, caring, or liking attribute to a consumer in a region- or country-specific manner. By way of illustration, a fragrance composition containing about 0.6 to 5.9 wt % Amber Xtreme™ in combination with about 0.2 to 2.0 wt % geraniol and/or about 2.5 to 4.9 wt % helional was perceived by consumers in both North America (e.g., the U.S.) and South America (e.g., Brazil) as imparting a caring attribute to the fragrance composition. Similarly, a fragrance composition containing 0.07 to 1.3 wt % Amber Xtreme™ in combination with 0.4 to 2.0 wt % citronellol and/or about 0.06 to 0.13 wt % damascenone was perceived by consumers in North America (e.g., the U.S.), South America (e.g., Brazil) and Asia (e.g., China) as imparting a fresh attribute to the fragrance composition. Accordingly, in one embodiment, a fragrance composition or consumer product containing about 0.6 to 5.9 wt % Amber Xtreme™ in combination with about 0.2 to 2.0 wt % geraniol and/or about 2.5 to 4.9 wt % helional, which imparts a caring attribute to a fragrance composition, is formulated for a consumer located in the Americas, in particular the U.S. and Brazil. In another embodiment, a fragrance composition or consumer product containing about 0.07 to 1.3 wt % Amber Xtreme™ in combination with about 0.4 to 2.0 wt % citronellol and/or about 0.06 to 0.13 wt % damascenone, which imparts a fresh attribute to a fragrance composition, is formulated for a consumer located in the Americas and/or Asia, in particular the U.S., Brazil and/or China. As used herein, the term “about” is intended to refer to an amount ±0.01-0.1% of the amount specified.

The fragrance compositions of the present invention are widely applicable to any perfumery product, including perfumes and colognes, the perfuming of personal care products such as soaps, shower gels, and hair care products, fabric care products, air fresheners, and cosmetic preparations. The present invention can also be used to perfume cleaning agents, such as, but not limited to, detergents, dishwashing materials, scrubbing compositions, window cleaners and the like. In these preparations, the fragrance compositions of the present invention can be used alone or in combination with other perfuming compositions, solvents, adjuvants and the like. The nature and variety of the other ingredients that can also be employed are known to those with skill in the art. Many types of additional fragrances can be employed in the present invention so long as the fragrance is complementary (i.e., compatible) with the other components being employed. Accordingly, in addition to Amber Xtreme™ and a synergistic fragrance ingredient, the fragrance composition of the invention may further include a complementary fragrance ingredient or compound. Optional complementary fragrances, if not already included as a synergistic fragrance ingredient, that can be used in the fragrance combinations of this invention, include but are not limited to:

i) hydrocarbons, such as, for example, 3-carene, α-pinene, β-pinene, α-terpinene, γ-terpinene, p-cymene, bisabolene, camphene, caryophyllene, cedrene, farnesene, limonene, longifolene, myrcene, ocimene, valencene, (E,Z)-1,3,5-undecatriene, styrene, and diphenylmethane;

ii) aliphatic alcohols, such as, for example, hexanol, octanol, 3-octanol, 2,6-dimethylheptanol, 2-methyl-2-heptanol, 2-methyl-2-octanol, (E)-2-hexenol, (E)- and (Z)-3-hexenol, 1-octen-3-ol, a mixture of 3,4,5,6,6-pentamethyl-3/4-hepten-2-ol and 3,5,6,6-tetramethyl-4-methyleneheptan-2-ol, (E,Z)-2,6-nonadienol, 3,7-dimethyl-7-methoxyoctan-2-ol, 9-decenol, 10-undecenol, 4-methyl-3-decen-5-ol, aliphatic aldehydes and their acetals such as for example hexanal, heptanal, octanal, nonanal, decanal, undecanal, dodecanal, tridecanal, 2-methyloctanal, 2-methylnonanal, (E)-2-hexenal, (Z)-4-heptenal, 2,6-dimethyl-5-heptenal, 10-undecenal, (E)-4-decenal, 2-dodecenal, 2,6,10-trimethyl-5,9-undecadienal, heptanal-diethylacetal, 1,1-dimethoxy-2,2,5-trimethyl-4-hexene, and citronellyl oxyacetaldehyde;

iii) aliphatic ketones and oximes thereof, such as, for example, 2-heptanone, 2-octanone, 3-octanone, 2-nonanone, 5-methyl-3-heptanone, 5-methyl-3-heptanone oxime, 2,4,4,7-tetramethyl-6-octen-3-one, aliphatic sulfur-containing compounds, such as for example 3-methylthiohexanol, 3-methylthiohexyl acetate, 3-mercaptohexanol, 3-mercaptohexyl acetate, 3-mercaptohexyl butyrate, 3-acetylthiohexyl acetate, 1-menthene-8-thiol, and aliphatic nitriles (e.g., 2-nonenenitrile, 2-tridecenenitrile, 2,12-tridecenenitrile, 3,7-dimethyl-2,6-octadienenitrile, and 3,7-dimethyl-6-octenenitrile);

iv) aliphatic carboxylic acids and esters thereof, such as, for example, (E)- and (Z)-3-hexenylformate, ethyl acetoacetate, isoamyl acetate, hexyl acetate, 3,5,5-trimethylhexyl acetate, 3-methyl-2-butenyl acetate, (E)-2-hexenyl acetate, (E)- and (Z)-3-hexenyl acetate, octyl acetate, 3-octyl acetate, 1-octen-3-yl acetate, ethyl butyrate, butyl butyrate, isoamyl butyrate, hexylbutyrate, (E)- and (Z)-3-hexenyl isobutyrate, hexyl crotonate, ethylisovalerate, ethyl-2-methyl pentanoate, ethyl hexanoate, allyl hexanoate, ethyl heptanoate, allyl heptanoate, ethyl octanoate, ethyl-(E,Z)-2,4-decadienoate, methyl-2-octinate, methyl-2-noninate, allyl-2-isoamyl oxyacetate, and methyl-3,7-dimethyl-2,6-octadienoate;

v) acyclic terpene alcohols, such as, for example, citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol 2,6-dimethyl-2,5,7-octatrien-1-ol; as well as formates, acetates, propionates, isobutyrates, butyrates, isovalerates, pentanoates, hexanoates, crotonates, tiglinates and 3-methyl-2-butenoates thereof;

vi) acyclic terpene aldehydes and ketones, such as, for example, geranial, neral, citronellal, 7-hydroxy-3,7-dimethyloctanal, 7-methoxy-3,7-dimethyloctanal, 2,6,10-trimethyl-9-undecenal, α-sinensal, β-sinensal, geranylacetone, as well as the dimethyl- and diethylacetals of geranial, neral and 7-hydroxy-3,7-dimethyloctanal;

vii) cyclic terpene alcohols, such as, for example, menthol, isopulegol, alpha-terpineol, terpinen-4-ol, menthan-8-ol, menthan-1-ol, menthan-7-ol, borneol, isoborneol, linalool oxide, nopol, cedrol, ambrinol, vetiverol, guaiol, and the formates, acetates, propionates, isobutyrates, butyrates, isovalerates, pentanoates, hexanoates, crotonates, tiglinates and 3-methyl-2-butenoates of alpha-terpineol, terpinen-4-ol, methan-8-ol, methan-1-ol, methan-7-ol, borneol, isoborneol, linalool oxide, nopol, cedrol, ambrinol, vetiverol, and guaiol;

viii) cyclic terpene aldehydes and ketones, such as, for example, menthone, isomenthone, 8-mercaptomenthan-3-one, carvone, camphor, fenchone, α-ionone, β-ionone, α-n-methylionone, β-n-methylionone, α-isomethylionone, β-isomethylionone, alpha-irone, α-damascone, β-damascone, β-damascenone, δ-damascone, γ-damascone, 1-(2,4,4-trimethyl-2-cyclohexen-1-yl)-2-buten-1-one, 1,3,4,6,7,8a-hexahydro-1,1,5,5-tetramethyl-2H-2,4a-methanonaphthalen-8(5H—)-one, nootkatone, dihydronootkatone; acetylated cedarwood oil (cedryl methyl ketone);

ix) cyclic alcohols, such as, for example, 4-tert-butylcyclohexanol, 3,3,5-trimethylcyclohexanol, 3-isocamphylcyclohexanol, 2,6,9-trimethyl-Z2,Z5,E9-cyclododecatrien-1-ol, 2-isobutyl-4-methyltetrahydro-2H-pyran-4-ol;

x) cycloaliphatic alcohols, such as, for example, alpha, 3,3-trimethylcyclo-hexylmethanol, 2-methyl-4-(2,2,3-trimethyl-3-cyclopent-1-yl)butanol, 2-methyl-4-(2,2,3-trimethyl-3-cyclopent-1-yl)-2-buten-1-ol, 2-ethyl-4-(2,2,3-trimethyl-3-cyclopent-1-yl)-2-buten-1-ol, 3-methyl-5-(2,2,3-trimethyl-3-cyclopent-1-yl)-pentan-2-ol, 3-methyl-5-(2,2,3-trimethyl-3-cyclopent-1-yl)-4-penten-2-ol, 3,3-dimethyl-5-(2,2,3-trimethyl-3-cyclopent-1-yl)-4-penten-2-ol, 1-(2,2,6-trimethylcyclohexyl)pentan-3-ol, 1-(2,2,6-trimethylcyclohexyl)hexan-3-ol;

xi) cyclic and cycloaliphatic ethers, such as, for example, cineole, cedryl methyl ether, cyclododecyl methyl ether;

xii) (ethoxymethoxy)cyclododecane; alpha-cedrene epoxide, 3a,6,6,9a-tetramethyldodecahydronaphtho[2,1-b]furan, 3a-ethyl-6,6,9a-trimethyldodecahydronaphtho[2,1-b]furan, 1,5,9-trimethyl-13-oxabicyclo[10.1.0]-trideca-4,8-diene, rose oxide, 2-(2,4-dimethyl-3-cyclohexen-1-yl)-5-methyl-5-(1-methylpropyl)-1,3-dioxan-;

xiii) cyclic ketones, such as, for example, 4-tert-butylcyclohexanone, 2,2,5-trimethyl-5-pentylcyclopentanone, 2-heptylcyclopentanone, 2-pentylcyclopentanone, 2-hydroxy-3-methyl-2-cyclopenten-1-one, 3-methyl-cis-2-penten-1-yl-2-cyclopenten-1-one, 3-methyl-2-pentyl-2-cyclopenten-1-one, 3-methyl-4-cyclopentadecenone, 3-methyl-5-cyclopentadecenone, 3-methylcyclopentadecanone, 4-(1-ethoxyvinyl)-3,3,5,5-tetramethylcyclohexanone, 4-tert-pentylcyclohexanone, 5-cyclohexadecen-1-one, 6,7-dihydro-1,1,2,3,3-pentamethyl-4(5H)-indanone, 5-cyclohexadecen-1-one, 8-cyclohexadecen-1-one, 9-cycloheptadecen-1-one, cyclopentadecanone, cycloaliphatic aldehydes, such as, for example, 2,4-dimethyl-3-cyclohexene carbaldehyde, 2-methyl-4-(2,2,6-trimethyl-cyclohexen-1-yl)-2-butenal, 4-(4-hydroxy-4-methylpentyl)-3-cyclohexene carbaldehyde, 4-(4-methyl-3-penten-1-yl)-3-cyclohexene carbaldehyde;

xiv) cycloaliphatic ketones, such as, for example, 1-(3,3-dimethylcyclohexyl)-4-penten-1-one, 1-(5,5-dimethyl-1-cyclohexen-1-yl)-4-penten-1-one, 2,3,8,8-tetramethyl-1,2,3,4,5,6,7,8-octahydro-2-naphtalenyl methyl-ketone, methyl-2,6,10-trimethyl-2,5,9-cyclododecatrienyl ketone, tert-butyl-(2,4-dimethyl-3-cyclohexen-1-yl)ketone;

xv) esters of cyclic alcohols, such as, for example, 2-tert-butylcyclohexyl acetate, 4-tert-butylcyclohexyl acetate, 2-tert-pentylcyclohexyl acetate, 4-tert-pentylcyclohexyl acetate, decahydro-2-naphthyl acetate, 3-pentyltetrahydro-2H-pyran-4-yl acetate, decahydro-2,5,5,8a-tetramethyl-2-naphthyl acetate, 4,7-methano-3a,4,5,6,7,7a-hexahydro-5 or 6-indenyl acetate, 4,7-methano-3a,4,5,6,7,7a-hexahydro-5 or 6-indenyl propionate, 4,7-methano-3a,4,5,6,7,7a-hexahydro-5 or 6-indenyl-isobutyrate, 4,7-methanooctahydro-5 or 6-indenyl acetate;

xvi) esters of cycloaliphatic carboxylic acids, such as, for example, allyl 3-cyclohexyl-propionate, allyl cyclohexyl oxyacetate, methyl dihydrojasmonate, methyl jasmonate, methyl 2-hexyl-3-oxocyclopentanecarboxylate, ethyl 2-ethyl-6,6-dimethyl-2-cyclohexenecarboxylate, ethyl 2,3,6,6-tetramethyl-2-cyclohexenecarboxylate, ethyl 2-methyl-1,3-dioxolane-2-acetate;

xvii) aromatic and aliphatic alcohols, such as, for example, benzyl alcohol, 1-phenylethyl alcohol, 2-phenylethyl alcohol, 3-phenylpropanol, 2-phenylpropanol, 2-phenoxyethanol, 2,2-dimethyl-3-phenylpropanol, 2,2-dimethyl-3-(3-methylphenyl)propanol, 1,1-dimethyl-2-phenylethyl alcohol, 1,1-dimethyl-3-phenylpropanol, 1-ethyl-1-methyl-3-phenylpropanol, 2-methyl-5-phenylpentanol, 3-methyl-5-phenylpentanol, 3-phenyl-2-propen-1-ol, 4-methoxybenzyl alcohol, 1-(4-isopropylphenyl)ethanol;

xviii) esters of aliphatic alcohols and aliphatic carboxylic acids, such as, for example, benzyl acetate, benzyl propionate, benzyl isobutyrate, benzyl isovalerate, 2-phenylethyl acetate, 2-phenylethyl propionate, 2-phenylethyl isobutyrate, 2-phenylethyl isovalerate, 1-phenylethyl acetate, α-trichloromethylbenzyl acetate, α,α-dimethylphenylethyl acetate, alpha, alpha-dimethylphenylethyl butyrate, cinnamyl acetate, 2-phenoxyethyl isobutyrate, 4-methoxybenzyl acetate, araliphatic ethers, such as for example 2-phenylethyl methyl ether, 2-phenylethyl isoamyl ether, 2-phenylethyl-1-ethoxyethyl ether, phenylacetaldehyde dimethyl acetal, phenylacetaldehyde diethyl acetal, hydratropaaldehyde dimethyl acetal, phenylacetaldehyde glycerol acetal, 2,4,6-trimethyl-4-phenyl-1,3-dioxane, 4,4a,5,9b-tetrahydroindeno[1,2-d]-m-dioxin, 4,4a,5,9b-tetrahydro-2,4-dimethylindeno[1,2-d]-m-dioxin;

xix) aromatic and aliphatic aldehydes, such as, for example, benzaldehyde; phenylacetaldehyde, 3-phenylpropanal, hydratropaldehyde, 4-methylbenzaldehyde, 4-methylphenylacetaldehyde, 3-(4-ethylphenyl)-2,2-dimethylpropanal, 2-methyl-3-(4-isopropylphenyl)propanal, 2-methyl-3-(4-Cert-butylphenyl)propanal, 3-(4-tert-butylphenyl)propanal, cinnamaldehyde, alpha-butylcinnamaldehyde, alpha-amylcinnamaldehyde, alpha-hexylcinnamaldehyde, 3-methyl-5-phenylpentanal, 4-methoxybenzaldehyde, 4-hydroxy-3-methoxybenzaldehyde, 4-hydroxy-3-ethoxybenzaldehyde, 3,4-methylene-dioxybenzaldehyde, 3,4-dimethoxybenzaldehyde, 2-methyl-3-(4-methoxyphenyl)propanal, 2-methyl-3-(4-methylendioxyphenyl)propanal;

xx) aromatic and aliphatic ketones, such as, for example, acetophenone, 4-methylacetophenone, 4-methoxyacetophenone, 4-tert-butyl-2,6-dimethylacetophenone, 4-phenyl-2-butanone, 4-(4-hydroxyphenyl)-2-butanone, 1-(2-naphthalenyl)ethanone, benzophenone, 1,1,2,3,3,6-hexamethyl-5-indanyl methyl ketone, 6-tert-butyl-1,1-dimethyl-4-indanyl methyl ketone, 1-[2,3-dihydro-1,1,2,6-tetramethyl-3-(1-methyl-ethyl)-1H-5-indenyl]ethanone, 5′,6′,7′,8′-tetrahydro-3′,5′,5′,6′,8′,8′-hexamethyl-2-acetonaphthone;

xxi) aromatic and araliphatic carboxylic acids and esters thereof, such as, for example, benzoic acid, phenylacetic acid, methyl benzoate, ethyl benzoate, hexyl benzoate, benzyl benzoate, methyl phenylacetate, ethyl phenylacetate, geranyl phenylacetate, phenylethyl phenylacetate, methyl cinnamate, ethyl cinnamate, benzyl cinnamate, phenylethyl cinnamate, cinnamyl cinnamate, allyl phenoxyacetate, methyl salicylate, isoamyl salicylate, hexyl salicylate, cyclohexyl salicylate, cis-3-hexenyl salicylate, benzyl salicylate, phenylethyl salicylate, methyl 2,4-dihydroxy-3,6-dimethylbenzoate, ethyl 3-phenylglycidate, ethyl 3-methyl-3-phenylglycidate;

xxii) nitrogen-containing aromatic compounds, such as, for example, 2,4,6-trinitro-1,3-dimethyl-5-tert-butylbenzene, 3,5-dinitro-2,6-dimethyl-4-tert-butylacetophenone, cinnamonitrile, 5-phenyl-3-methyl-2-pentenonitrile, 5-phenyl-3-methylpentanonitrile, methyl anthranilate, methy-N-methylanthranilate, Schiff's bases of methyl anthranilate with 7-hydroxy-3,7-dimethyloctanal, 2-methyl-3-(4-tert-butylphenyl)propanal or 2,4-dimethyl-3-cyclohexene carbaldehyde, 6-isopropylquinoline, 6-isobutylquinoline, 6-sec-butylquinoline, indole, skatole, 2-methoxy-3-isopropylpyrazine, 2-isobutyl-3-methoxypyrazine;

xxiii) phenols, phenyl ethers and phenyl esters, such as, for example, estragole, anethole, eugenol, eugenyl methyl ether, isoeugenol, isoeugenol methyl ether, thymol, carvacrol, diphenyl ether, beta-naphthyl methyl ether, beta-naphthyl ethyl ether, beta-naphthyl isobutyl ether, 1,4-dimethoxybenzene, eugenyl acetate, 2-methoxy-4-methylphenol, 2-ethoxy-5-(1-propenyl)phenol, p-cresyl phenylacetate;

xxiv) heterocyclic compounds, such as, for example, 2,5-dimethyl-4-hydroxy-2H-furan-3-one, 2-ethyl-4-hydroxy-5-methyl-2H-furan-3-one, 3-hydroxy-2-methyl-4H-pyran-4-one, 2-ethyl-3-hydroxy-4H-pyran-4-one;

xxv) lactones, such as, for example, 1,4-octanolide, 3-methyl-1,4-octanolide, 1,4-nonanolide, 1,4-decanolide, 8-decen-1,4-olide, 1,4-undecanolide, 1,4-dodecanolide, 1,5-decanolide, 1,5-dodecanolide, 1,15-pentadecanolide, cis- and trans-11-pentadecen-1,15-olide, cis- and trans-12-pentadecen-1,15-olide, 1,16-hexadecanolide, 9-hexadecen-1,16-olide, 10-oxa-1,16-hexadecanolide, 11-oxa-1,16-hexadecanolide, 12-oxa-1,16-hexadecanolide, ethylene-1,12-dodecanedioate, ethylene-1,13-tridecanedioate, coumarin, 2,3-dihydrocoumarin, and octahydrocoumarin; and

xxvi) essential oils, concretes, absolutes, resins, resinoids, balsams, tinctures such as for example ambergris tincture, amyris oil, angelica seed oil, angelica root oil, aniseed oil, valerian oil, basil oil, tree moss absolute, bay oil, armoise oil, benzoe resinoid, bergamot oil, beeswax absolute, birch tar oil, bitter almond oil, savory oil, buchu leaf oil, cabreuva oil, cade oil, calamus oil, camphor oil, cananga oil, cardamom oil, cascarilla oil, cassia oil, cassie absolute, castoreum absolute, cedar leaf oil, cedar wood oil, costus oil, citronella oil, lemon oil, copaiba balsam, copaiba balsam oil, coriander oil, costus root oil, cumin oil, cypress oil, davana oil, dill weed oil, dill seed oil, eau de brouts absolute, oakmoss absolute, elemi oil, estragon oil, eucalyptus citriodora oil, eucalyptus oil (cineole type), fennel oil, fir needle oil, galbanum oil, galbanum resin, geranium oil, grapefruit oil, guaiacwood oil, gurjun balsam, gurjun balsam oil, helichrysum absolute, helichrysum oil, ginger oil, iris root absolute, iris root oil, jasmine absolute, calamus oil, blue camomile oil, Roman camomile oil, carrot seed oil, cascarilla oil, pine needle oil, spearmint oil, caraway oil, labdanum oil, labdanum absolute, labdanum resin, lavandin absolute, lavandin oil, lavender absolute, lavender oil, lemon-grass oil, lovage oil, lime oil distilled, lime oil expressed, linaloe oil, Litsea cubeba oil, laurel leaf oil, mace oil, marjoram oil, mandarin oil, massoi (bark) oil, mimosa absolute, ambrette seed oil, musk tincture, clary sage oil, nutmeg oil, myrrh absolute, myrrh oil, myrtle oil, clove leaf oil, clove bud oil, neroli oil, olibanum absolute, olibanum oil, opopanax oil, orange flower absolute, orange oil, origanum oil, palmarosa oil, patchouli oil, perilla oil, Peru balsam oil, parsley leaf oil, parsley seed oil, petitgrain oil, peppermint oil, pepper oil, pimento oil, pine oil, pennyroyal oil, rose absolute, rosewood oil, rose oil, rosemary oil, Dalmatian sage oil, Spanish sage oil, sandal-wood oil, celery seed oil: spike-lavender oil, star anise oil, storax oil, tagetes oil, fir needle oil, tea tree oil, turpentine oil, thyme oil, Tolu balsam, tonka bean absolute, tuberose absolute, vanilla extract, violet leaf absolute, verbena oil, vetiver oil, juniper berry oil, wine lees oil, wormwood oil, wintergreen oil, ylang-ylang oil, hyssop oil, civet absolute, cinnamon leaf oil, cinnamon bark oil, and fractions thereof or ingredients isolated therefrom.

The amount of complementary fragrance ingredient used in the fragrance composition can be in the range of from about 0.001 wt % to about 50 wt %, or any range in therebetween. In certain embodiments, the complementary fragrance ingredient is, if not already included as a synergistic fragrance ingredient, one or more of ambrettolide, dipropylene glycol (CAS No. 110-98-5), 1-(1,2,3,5,6,7,8,8a-octahydro-2,3,8,8-tetramethyl-2-naphthyl)ethan-1-one (i.e., Iso E Super®; CAS No. 68155-66-8), or Kharismal (methyl 3-oxo-2-pentylcyclopentaneacetate; CAS No. 24851-98-7). In certain embodiments, a fragrance composition imparting a fresh attribute will include about 0.07 to 1.3 wt % Amber Xtreme™, about 0.4 to 2.0 wt % citronellol and/or about 0.06 to 0.13 wt % damascenone, and further include one or more of about 0.9 to 14.8 wt % ambrettolide, about 0.1 to 1.9 wt % dipropylene glycol, about 14.8 to 46.7 wt % Iso E Super®, or about 21.8 to 24.7 wt % kharismal. In other embodiments, a fragrance composition imparting a caring attribute will include about 0.6 to 5.9 wt % Amber Xtreme™, about 0.2 to 2.0 wt % geraniol and/or about 2.5 to 4.9 wt % helional, and further include one or more of about 0.9 to 14.8 wt % ambrettolide, about 0.1 to 1.9 wt % dipropylene glycol, about 14.8 to 46.7 wt % Iso E Super®, or about 21.8 to 24.7 wt % kharismal.

As indicated, a fragrance formulation of the present invention is well-suited for use in a variety of consumer products and can be used as a neat fragrance formulation (e.g., in a fine fragrance) or may be encapsulated and/or delivered using a suitable carrier material. Some well-known materials include, for example, polymers, oligomers, other non-polymers such as surfactants, emulsifiers, lipids including fats, waxes and phospholipids, organic oils, mineral oils, petrolatum, natural oils, perfume fixatives, fibers, starches, sugars and solid surface materials such as zeolite and silica.

In addition to the fragrance composition itself, the present invention provides consumer products containing the fragrance composition as well as methods of using the synergistic fragrance combinations of the present invention to impart fresh, caring or liking attributes to a thereby fragrance composition or consumer product. Consumer products may include, for example, perfumes, colognes, bar soaps, liquid soaps, shower gels, foam baths, cosmetics, skin care products such as creams, lotions and shaving products, hair care products for shampooing, rinsing, conditioning, bleaching, coloring, dyeing and styling, deodorants and antiperspirants, feminine care products such as tampons and feminine napkins, baby care products such as diapers, bibs and wipes, family care products such as bath tissues, facial tissues, paper handkerchiefs or paper towels, fabric products such as fabric softeners and fresheners, air care products such as air fresheners and fragrance delivery systems, cosmetic preparations, cleaning agents and disinfectants such as detergents, dishwashing materials, scrubbing compositions, glass and metal cleaners such as window cleaners, countertop cleaners, floor and carpet cleaners, toilet cleaners and bleach additives, washing agents such as all-purpose, heavy duty, and hand washing or fine fabric washing agents including laundry detergents and rinse additives, dental and oral hygiene products such as toothpastes, tooth gels, dental flosses, denture cleansers, denture adhesives, dentifrices, tooth whitening and mouthwashes, health care and nutritional products and food products such as snack and beverage products. The consumer product of the present invention is a consumer product that contains Amber Xtreme™ and at least one synergistic fragrance ingredient. In some embodiments, the consumer product of the invention contains Amber Xtreme™ and at least one synergistic fragrance ingredient, and further includes a complementary fragrance ingredient.

The invention is described in greater detail by the following non-limiting examples.

### Example 1: Evaluation of Fragrances Containing Amber Xtreme™

A plurality of fragrance compositions, each containing more than 20 fragrance ingredients, were prepared. Exemplary formulations included inter alia the fragrance ingredients listed in Table 2.

TABLE 2

| Fragrance | Wt % of Fragrance in Formulation | | |
|---|---|---|---|
| Ingredient | Formulation A | Formulation B | Formulation C |
| Amber Xtreme ™ | 0.6% | 1.3% | 0.2% |
| Ambrettolide | 14.8% | 19.0% | 0.9% |
| Citronellol | 0% | 0.6% | 0.4% |
| Damascenone | 0.12% | 0.13% | 0% |
| Dipropylene | 0.11% | 0.3% | 1.9% |
| Glycol | | | |
| Geraniol | 0.4% | 0% | 0% |
| Helional | 2.5% | 2.5% | 0% |
| Iso E Super | 14.8% | 21.3% | 46.7% |
| Kharismal | 24.7% | 25.3% | 21.8% |
| Muscemor | 0% | 0% | 1.9% |

The formulations were assessed using three test product applications: blind sniff test with cotton; blind sniff test in beads wrapped into a dry towel; and blind sniff test in dilution. A panel of approximately 100 women consumers (18-65 years of age) from each of Brazil, China, the United Kingdom, France and the United States were presented with 90 fragrances over three sessions, i.e., during one session the 100 consumers smelled 30 fragrances. The consumers were allowed to smell each formulation as many times as they wished during the evaluation session.

The consumers were requested to give each of the formulations a score of overall liking or appreciation according to the following scale:

| | | | Neither | | | |
|---|---|---|---|---|---|---|
| Dislike | | | Like | | | Like |
| Very | | Dislike | Nor | Like | | Very |
| Much | Dislike | Slightly | dislike | Slightly | Like | Much |
| 1 | 2 | 3 | 4 | 5 | 6 | 7 |

By way of illustration, the liking scores for consumers in Brazil, China, France, the UK and the USA who evaluated exemplary Formulations A, B and C (Table 2) are presented in Table 3.

TABLE 3

| Score | 1 | 2 | 3 | 4 | 5 | 6 | 7 |
|---|---|---|---|---|---|---|---|
| | # of Consumers in Brazil | | | | | | |
| Formulation A | 5 | 7 | 8 | 13 | 19 | 27 | 27 |
| Formulation B | 4 | 9 | 7 | 7 | 16 | 26 | 37 |
| Formulation C | 6 | 9 | 3 | 7 | 24 | 26 | 31 |
| | # of Consumers in China | | | | | | |
| Formulation A | | 4 | 7 | 8 | 24 | 42 | 15 |
| Formulation B | 1 | 1 | 7 | 15 | 22 | 33 | 21 |
| Formulation C | | 2 | 4 | 14 | 36 | 30 | 14 |
| | # of Consumers in France | | | | | | |
| Formulation A | 4 | 3 | 10 | 10 | 28 | 31 | 15 |
| Formulation B | 9 | 4 | 8 | 7 | 24 | 32 | 17 |
| Formulation C | 5 | 8 | 6 | 7 | 29 | 32 | 14 |
| | # of Consumers in the UK | | | | | | |
| Formulation A | 5 | 9 | 13 | 12 | 22 | 20 | 20 |
| Formulation B | 5 | 12 | 10 | 17 | 19 | 22 | 16 |
| Formulation C | 2 | 10 | 12 | 14 | 18 | 32 | 13 |
| USA | # of Consumers the USA | | | | | | |
| Formulation A | 5 | 3 | 9 | 4 | 27 | 24 | 28 |
| Formulation B | 8 | 7 | 8 | 7 | 18 | 30 | 22 |
| Formulation C | 8 | 9 | 8 | 13 | 29 | 20 | 13 |

Further, the consumers were asked to group each of the 30 fragrances into 3-10 groups (with each fragrance being a member of only one group) based on how the fragrances smelled, i.e., grouping of fragrances perceived to be similar to each other. After the groups were created, the consumer was asked to describe the different groups in terms of their attributes or benefits, e.g., does the scent make you feel fresh.

The data received from the consumer evaluations were collected and analyzed to identify synergies between any two ingredients (and their amounts) in the formulations and the consumer benefits associated therewith. In particular, analysis was conducted on the consumer benefits of liking, caring, or fresh. Three different types of synergies were identified: negative synergy (representing a risk); neutral synergy (indicating that the ingredients were independent); and positive synergy (representing a benefit). Based upon this analysis, a set of rules for two ingredient combinations was determined for consumer benefits of liking, caring and fresh attributes.

### Example 2: Rules for Fragrances in Combination with Amber Xtreme™

From the 90 formulae tested, the highest scoring formulae from each of Brazil, China, the United Kingdom, France and the United States with consumer benefits of liking, caring, and fresh were identified. These top formulae from each country were analyzed to identify synergies (referred to as lift) between Amber Xtreme™ and a second ingredient in the formulations to understand the basis of the benefits of liking, fresh and caring. The rules generated for positive synergies between Amber Xtreme™ and a second fragrance in imparting caring, fresh and liking attributes are presented in Tables 4, 5 and 6, respectively. The tables provide the amount of Amber Xtreme™ in the formulation (range based on 1000 parts), the second fragrance ingredient and its amount (range based on 1000 parts) in the formulation, the ratio of Amber Xtreme™ to second fragrance ingredient and lift scores in each country. Notably, lift scores higher than 1.10 indicate that the rule is good for this variable, whereas lift scores lower than 1.10 indicate that the rule does not apply to this variable.

TABLE 4

| Amount of | | Amount of | | | | | | |
|---|---|---|---|---|---|---|---|---|
| Amber | Second | Second | | Lift Scores | | | | |
| Xtreme | Fragrance | Fragrance | Ratio | BR | CN | FR | UK | US |
| 1.85-19.74 | Ambrettolide | 9.26-189.87 | 1:11 | 1.16 | 1.10 | 1.11 | 1.03 | 1.14 |
| 1.85-19.74 | Ambrettolide | 9.26-148.15 | 1:10 | 1.14 | 1.08 | 1.13 | 0.97 | 1.09 |
| 0.43-19.74 | Ambrettolide | 9.26-105.26 | 1:8 | 1.08 | 1.06 | 1.02 | 0.93 | 1.06 |
| 0.43-12.66 | Ambrettolide | 9.26-189.87 | 1:16 | 1.12 | 1.09 | 1.02 | 1.04 | 1.18 |
| 0.43-19.74 | Ambrettolide | 34.50-148.15 | 1:11 | 1.15 | 1.04 | 0.99 | 0.87 | 1.10 |
| 0.43-7.35 | Ambrettolide | 34.50-198.62 | 1:19 | 1.13 | 1.06 | 0.97 | 1.00 | 1.20 |
| 0.43-7.35 | Ambrettolide | 9.26-148.15 | 1:17 | 1.09 | 1.06 | 1.01 | 0.99 | 1.14 |
| 0.43-12.66 | Ambrettolide | 34.50-198.62 | 1:22 | 1.10 | 1.03 | 0.94 | 0.93 | 1.16 |
| 1.85-12.66 | Citronellol | 3.70-10.00 | 1:1 | 1.00 | 1.10 | 0.98 | 1.10 | 1.06 |
| 1.85-12.66 | Citronellol | 3.70-20.69 | 1:1 | 1.00 | 1.08 | 0.97 | 1.06 | 1.09 |
| 1.85-7.35 | Citronellol | 3.70-20.69 | 1:2 | 0.94 | 1.05 | 0.95 | 1.00 | 1.01 |
| 6.17-59.11 | Damascenone | 0.97-1.27 | 19:1 | 1.11 | 1.00 | 0.95 | 0.95 | 1.14 |
| 5.00-59.11 | Damascenone | 0.99-1.27 | 18:1 | 1.02 | 0.99 | 0.86 | 0.93 | 1.01 |
| 0.68-59.11 | Damascenone | 0.55-1.23 | 16:1 | 0.97 | 0.95 | 0.87 | 0.85 | 0.99 |
| 0.68-12.66 | Damascenone | 0.55-1.27 | 6:1 | 1.04 | 1.03 | 0.95 | 0.95 | 1.10 |
| 0.68-7.35 | Geraniol | 1.38-30.00 | 1:1 | 1.01 | 1.02 | 0.94 | 0.94 | 1.06 |
| 0.68-19.74 | Geraniol | 2.94-19.74 | 1:1 | 1.15 | 1.04 | 1.07 | 0.91 | 1.09 |
| 5.00-19.74 | Geraniol | 2.94-30.00 | 1:1 | 1.06 | 1.04 | 0.97 | 0.89 | 0.99 |
| 0.68-7.35 | Geraniol | 1.38-5.48 | 2:1 | 1.10 | 1.03 | 1.02 | 0.97 | 1.15 |
| 6.17-19.74 | Geraniol | 1.38-19.74 | 1:1 | 1.15 | 1.05 | 1.06 | 0.91 | 1.11 |
| 0.68-19.74 | Geraniol | 1.38-19.74 | 1:1 | 1.12 | 1.03 | 1.05 | 0.91 | 1.10 |
| 13.70-72.31 | Helional | 0.68-19.74 | 1:4 | 1.17 | 1.07 | 1.07 | 0.98 | 1.14 |
| 13.70-29.41 | Helional | 0.68-12.66 | 1:3 | 1.16 | 1.08 | 1.05 | 1.06 | 1.20 |
| 17.25-49.26 | Helional | 0.43-59.11 | 1:2 | 1.11 | 1.01 | 0.91 | 0.97 | 1.12 |
| 24.69-49.26 | Helional | 6.17-59.11 | 1:2 | 1.14 | 1.02 | 0.97 | 1.02 | 1.12 |
| 17.25-72.31 | Helional | 0.43-59.11 | 1:2 | 1.13 | 1.02 | 0.95 | 0.92 | 1.09 |
| 13.70-25.32 | Helional | 0.43-12.66 | 1:4 | 1.14 | 1.04 | 0.97 | 0.95 | 1.20 |
| 13.70-29.41 | Helional | 0.43-7.35 | 1:6 | 1.10 | 1.02 | 0.95 | 0.93 | 1.14 |
| 13.70-72.31 | Helional | 0.68-59.11 | 1:2 | 1.13 | 1.02 | 1.00 | 0.94 | 1.08 |
| 24.69-72.31 | Helional | 6.17-59.11 | 1:2 | 1.15 | 1.03 | 1.00 | 0.95 | 1.09 |
| 13.70-49.26 | Helional | 0.68-59.11 | 1:2 | 1.11 | 1.01 | 0.97 | 1.00 | 1.11 |
| 5.00-164.74 | Hexenyl Cis-3- | 23.00-82.37 | 1:1 | 0.97 | 0.94 | 0.83 | 0.89 | 0.87 |
| | Salicylate | | | | | | | |
| 0.43-7.35 | Muscemor | 18.52-129.37 | 1:22 | 1.03 | 1.04 | 0.99 | 0.99 | 1.10 |
| 6.90-164.74 | Muscemor | 4.94-137.93 | 1:1 | 1.06 | 0.95 | 0.93 | 0.86 | 0.92 |
| 0.68-164.74 | Muscemor | 4.94-41.10 | 2:1 | 1.00 | 0.96 | 0.97 | 0.97 | 0.93 |
| 0.43-19.74 | Muscemor | 41.10-137.93 | 1:13 | 1.07 | 1.00 | 0.95 | 0.80 | 1.08 |
| 0.68-7.35 | Muscemor | 18.52-137.93 | 1:14 | 1.03 | 1.05 | 1.06 | 1.02 | 1.11 |
| 1.85-19.74 | Muscemor | 18.52-137.93 | 1:7 | 1.08 | 1.08 | 1.09 | 0.97 | 1.08 |
| 0.43-7.35 | Muscemor | 18.52-137.93 | 1:21 | 1.03 | 1.03 | 0.98 | 0.97 | 1.11 |
| 1.85-164.74 | Muscemor | 4.94-137.93 | 1:1 | 1.04 | 0.99 | 0.99 | 0.91 | 0.95 |
| 0.43-19.74 | Muscemor | 36.76-129.37 | 1:10 | 1.09 | 1.02 | 0.97 | 0.87 | 1.06 |
| 0.43-7.35 | Muscemor | 36.76-137.93 | 1:22 | 1.04 | 1.01 | 0.92 | 0.93 | 1.12 |
| 0.68-19.74 | Muscemor | 18.52-65.79 | 1:5 | 1.08 | 1.06 | 1.10 | 0.97 | 1.05 |
| 1.85-164.74 | Muscemor | 4.94-65.79 | 2:1 | 1.04 | 0.98 | 1.00 | 0.91 | 0.89 |
| 1.85-19.74 | Veridian | 0.01-50.00 | 1:1 | 1.02 | 1.08 | 1.01 | 0.97 | 1.01 |
| 1.85-19.74 | Orange Flower | 1.32-5.00 | 3:1 | 0.99 | 1.06 | 1.00 | 0.95 | 0.95 |

The results of this analysis indicated that when a fragrance formulation includes Amber Xtreme™ and the benefit to be achieved is a caring attribute, the amount of Amber Xtreme™ in the formulation should be in the range of 0.6 to 5.9 wt % and the fragrance should also include about 0.2 to 2.0 wt % geraniol, and/or about 2.5 to 4.9 wt % helional. More specifically, a fragrance composition containing 0.6 to 5.9 wt % Amber Xtreme™ in combination with 0.2 to 2.0 wt % geraniol, and/or about 2.5 to 4.9 wt % helional was perceived by consumers in both North America and South America as imparting a caring attribute to the fragrance composition.

TABLE 5

| Amount of | | Amount of | | | | | | |
|---|---|---|---|---|---|---|---|---|
| Amber | Second | Second | | Lift Scores | | | | |
| Xtreme | Fragrance | Fragrance | Ratio | BR | CN | FR | UK | US |
| 1.85-19.74 | Ambrettolide | 9.26-189.87 | 1:11 | 1.23 | 1.15 | 1.17 | 1.04 | 1.23 |
| 1.85-19.74 | Ambrettolide | 9.26-148.15 | 1:10 | 1.20 | 1.14 | 1.10 | 0.99 | 1.13 |
| 0.43-19.74 | Ambrettolide | 9.26-105.26 | 1:8 | 1.10 | 1.10 | 0.99 | 0.81 | 1.03 |
| 0.43-12.66 | Ambrettolide | 9.26-189.87 | 1:16 | 1.25 | 1.13 | 1.07 | 1.00 | 1.15 |
| 0.43-19.74 | Ambrettolide | 34.50-148.15 | 1:11 | 1.10 | 1.13 | 0.96 | 0.86 | 0.98 |
| 0.43-7.35 | Ambrettolide | 34.50-198.62 | 1:19 | 1.24 | 1.15 | 1.02 | 0.94 | 1.15 |
| 0.43-7.35 | Ambrettolide | 9.26-148.15 | 1:17 | 1.24 | 1.12 | 0.98 | 0.94 | 1.03 |
| 0.43-12.66 | Ambrettolide | 34.50-198.62 | 1:22 | 1.21 | 1.14 | 0.91 | 0.87 | 1.03 |
| 1.85-12.66 | Citronellol | 3.70-10.00 | 1:1 | 1.15 | 1.13 | 1.04 | 0.99 | 1.17 |
| 1.85-12.66 | Citronellol | 3.70-20.69 | 1:1 | 1.19 | 1.13 | 1.03 | 0.96 | 1.18 |
| 1.85-7.35 | Citronellol | 3.70-20.69 | 1:2 | 1.15 | 1.12 | 0.93 | 0.89 | 1.07 |
| 6.17-59.11 | Damascenone | 0.97-1.27 | 19:1 | 1.25 | 1.04 | 1.07 | 1.04 | 1.15 |
| 5.00-59.11 | Damascenone | 0.99-1.27 | 18:1 | 1.10 | 1.00 | 0.97 | 1.04 | 0.99 |
| 0.68-59.11 | Damascenone | 0.55-1.23 | 16:1 | 1.09 | 1.01 | 0.87 | 0.94 | 0.94 |
| 0.68-12.66 | Damascenone | 0.55-1.27 | 6:1 | 1.18 | 1.11 | 0.99 | 1.01 | 1.13 |
| 0.68-7.35 | Geraniol | 1.38-30.00 | 1:1 | 1.13 | 1.13 | 0.90 | 0.94 | 1.04 |
| 0.68-19.74 | Geraniol | 2.94-19.74 | 1:1 | 1.12 | 1.16 | 1.03 | 0.96 | 1.10 |
| 5.00-19.74 | Geraniol | 2.94-30.00 | 1:1 | 1.03 | 1.13 | 0.94 | 0.92 | 0.96 |
| 0.68-7.35 | Geraniol | 1.38-5.48 | 2:1 | 1.24 | 1.17 | 0.99 | 0.98 | 1.15 |
| 6.17-19.74 | Geraniol | 1.38-19.74 | 1:1 | 1.18 | 1.16 | 1.04 | 0.92 | 1.13 |
| 0.68-19.74 | Geraniol | 1.38-19.74 | 1:1 | 1.16 | 1.15 | 1.02 | 0.93 | 1.13 |
| 13.70-72.37 | Helional | 0.68-19.74 | 1:4 | 1.16 | 1.16 | 1.11 | 1.01 | 1.20 |
| 13.70-29.41 | Helional | 0.68-12.66 | 1:3 | 1.24 | 1.19 | 1.10 | 1.08 | 1.25 |
| 17.25-49.26 | Helional | 0.43-59.11 | 1:2 | 1.15 | 1.06 | 0.98 | 0.96 | 1.04 |
| 24.69-49.26 | Helional | 6.17-59.11 | 1:2 | 1.19 | 1.08 | 1.07 | 1.06 | 1.14 |
| 17.25-72.37 | Helional | 0.43-59.11 | 1:2 | 1.10 | 1.06 | 1.01 | 0.92 | 1.04 |
| 13.70-25.32 | Helional | 0.43-12.66 | 1:4 | 1.20 | 1.10 | 1.02 | 0.99 | 1.11 |
| 13.70-29.41 | Helional | 0.43-7.35 | 1:6 | 1.15 | 1.13 | 0.90 | 0.91 | 1.00 |
| 13.70-72.37 | Helional | 0.68-59.11 | 1:2 | 1.12 | 1.08 | 1.06 | 0.99 | 1.12 |
| 24.69-72.37 | Helional | 6.17-59.11 | 1:2 | 1.13 | 1.08 | 1.09 | 1.00 | 1.12 |
| 13.70-49.26 | Helional | 0.68-59.11 | 1:2 | 1.17 | 1.09 | 1.04 | 1.04 | 1.14 |
| 5.00-164.74 | Hexenyl Cis- | 23.00-82.37 | 1:1 | 0.98 | 0.99 | 0.75 | 0.94 | 0.84 |
| | 3-Salicylate | | | | | | | |
| 0.43-7.35 | Muscemor | 18.52-129.37 | 1:22 | 1.15 | 1.11 | 0.94 | p.86 | 1.06 |
| 6.90-164.74 | Muscemor | 4.94-137.93 | 1:1 | 0.99 | 1.01 | 0.86 | 0.81 | 1.00 |
| 0.68-164.74 | Muscemor | 4.94-41.10 | 2:1 | 1.06 | 0.99 | 0.87 | 0.92 | 1.03 |
| 0.43-19.74 | Muscemor | 41.10-137.93 | 1:13 | 1.06 | 1.07 | 0.91 | 0.76 | 1.02 |
| 0.68-7.35 | Muscemor | 18.52-137.93 | 1:14 | 1.24 | 1.15 | 1.02 | 0.92 | 1.21 |
| 1.85-19.74 | Muscemor | 18.52-137.93 | 1:7 | 1.18 | 1.14 | 1.07 | 0.87 | 1.18 |
| 0.43-7.35 | Muscemor | 18.52-137.93 | 1:21 | 1.19 | 1.12 | 0.94 | 0.85 | 1.10 |
| 1.85-164.74 | Muscemor | 4.94-137.93 | 1:1 | 1.07 | 1.01 | 0.93 | 0.86 | 1.05 |
| 0.43-19.74 | Muscemor | 36.76-129.37 | 1:10 | 1.01 | 1.12 | 0.91 | 0.77 | 1.00 |
| 0.43-7.35 | Muscemor | 36.76-137.93 | 1:22 | 1.13 | 1.13 | 0.87 | 0.79 | 1.06 |
| 0.68-19.74 | Muscemor | 18.52-65.79 | 1:5 | 1.12 | 1.13 | 1.06 | 0.91 | 1.15 |
| 1.85-164.74 | Muscemor | 4.94-65.79 | 2:1 | 1.01 | 0.99 | 0.93 | 0.87 | 1.00 |
| 1.85-19.74 | Veridian | 0.01-50.00 | 1:1 | 1.08 | 1.07 | 1.08 | 0.96 | 1.13 |
| 1.85-19.74 | Orange Flower | 1.32-5.00 | 3:1 | 1.03 | 1.10 | 0.97 | 0.87 | 1.02 |

The results of this analysis indicated that when a fragrance formulation includes Amber Xtreme™ and the benefit to be achieved is a fresh attribute in a majority of countries, the amount of Amber Xtreme™ in the formulation be in the range of 0.07 to 1.3 wt % and the fragrance should also include about 0.4 to 2.0 wt % citronellol, and/or about 0.06 to 0.13 wt % damascenone. More specifically, a fragrance composition containing 0.07 to 1.3 wt % Amber Xtreme™ in combination with 0.4 to 2.0 wt % citronellol, and/or about 0.06 to 0.13 wt % damascenone was perceived by consumers in North America, South America and Asia as imparting a fresh attribute to the fragrance composition.

TABLE 6

| Amount of | | Amount of | | | | | | |
|---|---|---|---|---|---|---|---|---|
| Amber | Second | Second | | Lift Scores | | | | |
| Xtreme | Fragrance | Fragrance | Ratio | BR | CN | FR | UK | US |
| 1.85-19.74 | Ambrettolide | 9.26-189.87 | 1:11 | 1.69 | 1.33 | 1.27 | 1.21 | 1.54 |
| 1.85-19.74 | Ambrettolide | 9.26-148.15 | 1:10 | 1.60 | 1.29 | 1.22 | 1.20 | 1.43 |
| 0.43-19.74 | Ambrettolide | 9.26-105.26 | 1:8 | 1.37 | 1.16 | 1.04 | 1.02 | 0.93 |
| 0.43-12.66 | Ambrettolide | 9.26-189.87 | 1:16 | 1.58 | 1.29 | 1.23 | 1.15 | 1.43 |
| 0.43-19.74 | Ambrettolide | 34.50-148.15 | 1:11 | 1.32 | 1.25 | 1.04 | 0.98 | 1.26 |
| 0.43-7.35 | Ambrettolide | 34.50-198.62 | 1:19 | 1.47 | 1.31 | 1.17 | 1.02 | 1.39 |
| 0.43-7.35 | Ambrettolide | 9.26-148.15 | 1:17 | 1.46 | 1.25 | 1.17 | 1.12 | 1.30 |
| 0.43-12.66 | Ambrettolide | 34.50-198.62 | 1:22 | 1.32 | 1.27 | 1.09 | 0.96 | 1.26 |
| 1.85-12.66 | Citronellol | 3.70-10.00 | 1:1 | 1.58 | 1.09 | 1.08 | 1.13 | 1.13 |
| 1.85-12.66 | Citronellol | 3.70-20.69 | 1:1 | 1.50 | 1.13 | 1.07 | 1.07 | 1.09 |
| 1.85-7.35 | Citronellol | 3.70-20.69 | 1:2 | 1.36 | 1.05 | 0.97 | 1.03 | 0.88 |
| 6.17-59.11 | Damascenone | 0.97-1.27 | 19:1 | 1.37 | 1.23 | 1.15 | 1.06 | 1.48 |
| 5.00-59.11 | Damascenone | 0.99-1.27 | 18:1 | 1.22 | 1.01 | 0.94 | 1.02 | 1.30 |
| 0.68-59.11 | Damascenone | 0.55-1.23 | 16:1 | 0.94 | 1.05 | 0.90 | 0.99 | 1.11 |
| 0.68-12.66 | Damascenone | 0.55-1.27 | 6:1 | 1.21 | 1.23 | 1.06 | 1.08 | 1.39 |
| 0.68-7.35 | Geraniol | 1.38-30.00 | 1:1 | 1.19 | 1.18 | 1.01 | 1.05 | 1.24 |
| 0.68-19.74 | Geraniol | 2.94-19.74 | 1:1 | 1.34 | 1.33 | 1.15 | 1.15 | 1.48 |
| 5.00-19.74 | Geraniol | 2.94-30.00 | 1:1 | 1.30 | 1.11 | 0.93 | 1.00 | 1.21 |
| 0.68-7.35 | Geraniol | 1.38-5.48 | 2:1 | 1.34 | 1.35 | 1.20 | 1.14 | 1.48 |
| 6.17-19.74 | Geraniol | 1.38-19.74 | 1:1 | 1.46 | 1.32 | 1.13 | 1.05 | 1.39 |
| 0.68-19.74 | Geraniol | 1.38-19.74 | 1:1 | 1.31 | 1.32 | 1.14 | 1.09 | 1.38 |
| 13.70-72.37 | Helional | 0.68-19.74 | 1:4 | 1.49 | 1.36 | 1.22 | 1.17 | 1.57 |
| 13.70-29.41 | Helional | 0.68-12.66 | 1:3 | 1.57 | 1.40 | 1.31 | 1.23 | 1.72 |
| 17.25-49.26 | Helional | 0.43-59.11 | 1:2 | 1.38 | 1.17 | 1.09 | 1.01 | 1.31 |
| 24.69-49.26 | Helional | 6.17-59.11 | 1:2 | 1.57 | 1.20 | 1.19 | 1.12 | 1.52 |
| 17.25-72.37 | Helional | 0.43-59.11 | 1:2 | 1.34 | 1.17 | 1.05 | 1.00 | 1.25 |
| 13.70-25.32 | Helional | 0.43-12.66 | 1:4 | 1.23 | 1.34 | 1.18 | 1.11 | 1.54 |
| 13.70-29.41 | Helional | 0.43-7.35 | 1:6 | 1.20 | 1.28 | 1.11 | 1.07 | 1.35 |
| 13.70-72.37 | Helional | 0.68-59.11 | 1:2 | 1.36 | 1.23 | 1.13 | 1.11 | 1.40 |
| 24.69-72.37 | Helional | 6.17-59.11 | 1:2 | 1.49 | 1.20 | 1.12 | 1.08 | 1.41 |
| 13.70-49.26 | Helional | 0.68-59.11 | 1:2 | 1.40 | 1.23 | 1.18 | 1.15 | 1.48 |
| 5.00-164.74 | Hexenyl Cis- | 23.00-82.37 | 1:1 | 1.13 | 0.88 | 0.82 | 0.89 | 0.99 |
| | 3-Salicylate | | | | | | | |
| 0.43-7.35 | Muscemor | 18.52-129.37 | 1:22 | 1.26 | 1.19 | 1.11 | 1.11 | 1.01 |
| 6.90-164.74 | Muscemor | 4.94-137.93 | 1:1 | 1.20 | 1.00 | 0.90 | 0.83 | 0.79 |
| 0.68-164.74 | Muscemor | 4.94-41.10 | 2:1 | 1.23 | 1.01 | 1.05 | 1.08 | 0.93 |
| 0.43-19.74 | Muscemor | 41.10-137.93 | 1:13 | 0.92 | 1.21 | 0.94 | 0.91 | 0.93 |
| 0.68-7.35 | Muscemor | 18.52-137.93 | 1:14 | 1.40 | 1.27 | 1.20 | 1.18 | 1.15 |
| 1.85-19.74 | Muscemor | 18.52-137.93 | 1:7 | 1.51 | 1.23 | 1.13 | 1.09 | 1.06 |
| 0.43-7.35 | Muscemor | 18.52-137.93 | 1:21 | 1.24 | 1.21 | 1.10 | 1.06 | 1.01 |
| 1.85-164.74 | Muscemor | 4.94-137.93 | 1:1 | 1.31 | 1.04 | 1.00 | 0.96 | 0.86 |
| 0.43-19.74 | Muscemor | 36.76-129.37 | 1:10 | 1.12 | 1.19 | 0.98 | 0.97 | 0.97 |
| 0.43-7.35 | Muscemor | 36.76-137.93 | 1:22 | 1.12 | 1.22 | 1.03 | 0.95 | 0.97 |
| 0.68-19.74 | Muscemor | 18.52-65.79 | 1:5 | 1.40 | 1.24 | 1.15 | 1.19 | 1.15 |
| 1.85-164.74 | Muscemor | 4.94-65.79 | 2:1 | 1.34 | 0.97 | 0.98 | 0.99 | 0.84 |
| 1.85-19.74 | Veridian | 0.01-50.00 | 1:1 | 1.39 | 1.08 | 1.00 | 1.09 | 1.08 |
| 1.85-19.74 | Orange Flower | 1.32-5.00 | 3:1 | 1.36 | 1.02 | 0.93 | 1.05 | 0.88 |

The results of this analysis indicated that when a fragrance formulation includes Amber Xtreme™ and the benefit to be achieved is a liking attribute in a majority of countries, the amount of Amber Xtreme™ in the formulation should be in the range of 0.05 to 2.0 wt % and the fragrance should also include about 0.1 to 0.6 wt % geraniol, about 1.3 to 3.0 wt % helional, about 1.8 to 13.8 wt % muscemor, and/or about 0.9 to 19.0 wt % ambrettolide. More specifically, a fragrance composition containing 0.05 to 2.0 wt % Amber Xtreme™ in combination with 0.1 to 0.6 wt % geraniol, about 1.3 to 3.0 wt % helional, about 1.8 to 13.8 wt % muscemor, and/or about 0.9 to 19.0 wt % ambrettolide was perceived by consumers in North America, South America, Europe and Asia as imparting a liking attribute to the fragrance composition.

In addition to positive synergies, the above-referenced data demonstrate negative synergies as well. For example, whereas 0.5-16.47 wt % Amber Xtreme™ in combination with 2.30-8.24 wt % hexenyl cis-3-salicylate provided a positive lift score in Brazil for liking, this same combination of fragrances did not provide positive lift scores in the U.S., China, France or the U.K. for liking and failed to provide positive lift scores in any country for either caring or fresh attribute. Further, whereas 0.07-1.27 wt % Amber Xtreme™ in combination with 0.06-0.13 wt % damascenone provided a positive synergy in Brazil, China and the U.S. for the liking attribute, the use of a broader range of Amber Xtreme™, i.e., 0.50-5.91 wt %, in combination with a narrower range of damascenone, i.e., 0.10-0.13 wt % failed to provide positive lift scores in UK, France and China for this attribute. Therefore, the data provided herein demonstrate that the consumer as well as the combination of fragrance ingredients and the amount of fragrance ingredients each have an impact on perceived consumer benefits.

What is claimed is:

1. A fragrance composition for imparting a fresh attribute comprising
(a) about 0.07 to 1.3 wt % Amber Xtreme, and
at least one of the following:
(b) about 0.4 to 2.0 wt % citronellol,
(c) about 0.06 to 0.13 wt % damascenone.

2. The fragrance composition of claim 1, further comprising one or more of:
(d) about 0.9 to 14.8 wt % ambrettolide,
(e) about 0.1 to 1.9 wt % dipropylene glycol,
(f) about 14.8 to 46.7 wt % 1-(1,2,3,5,6,7,8,8a-octahydro-2,3,8,8-tetramethyl-2-naphthyl)ethan-1-one, or
(g) about 21.8 to 24.7 wt % kharismal.

3. A consumer product comprising the fragrance composition of claim 1.

4. A method for imparting a fresh attribute to a fragrance composition or consumer product comprising adding to the fragrance composition or consumer product:
(a) about 0.07 to 1.3 wt % Amber Xtreme, and
at least one of the following:
(b) about 0.4 to 2.0 wt % citronellol,
(c) about 0.06 to 0.13 wt % damascenone,
thereby imparting a fresh attribute to the fragrance composition or consumer product.

5. The method of claim 4, further comprising adding one or more of:
(d) about 0.9 to 14.8 wt % ambrettolide,
(e) about 0.1 to 1.9 wt % Dipropylene Glycol,
(f) about 14.8 to 46.7 wt % 1-(1,2,3,5,6,7,8,8a-octahydro-2,3,8,8-tetramethyl-2-naphthyl)ethan-1-one, or
(g) about 21.8 to 24.7 wt % kharismal.

6. A fragrance composition for imparting a caring attribute comprising
(a) about 0.6 to 5.9 wt % Amber Xtreme, and
at least one of the following:
(b) about 0.2 to 2.0 wt % geraniol,
(c) about 2.5 to 4.9 wt % helional.

7. The fragrance composition of claim 6, further comprising one or more of:
(d) about 0.9 to 14.8 wt % ambrettolide,
(e) about 0.1 to 1.9 wt % dipropylene glycol,
(f) about 14.8 to 46.7 wt % 1-(1,2,3,5,6,7,8,8a-octahydro-2,3,8,8-tetramethyl-2-naphthyl)ethan-1-one, or
(g) about 21.8 to 24.7 wt % kharismal.

8. A consumer product comprising the fragrance composition of claim 6.

9. The consumer product of claim 8, wherein said product is formulated for a consumer in North America or South America.

10. A method for imparting a caring attribute to a fragrance composition or consumer product comprising adding to the fragrance composition or consumer product:
(a) about 0.6 to 5.9 wt % Amber Xtreme, and
at least one of the following:
(b) about 0.2 to 2.0 wt % geraniol,
(c) about 2.5 to 4.9 wt % helional,
thereby imparting a caring attribute to the fragrance composition or consumer product.

11. The method of claim 10, wherein the method further comprises adding one or more of:
(d) about 0.9 to 14.8 wt % ambrettolide,
(e) about 0.1 to 1.9 wt % dipropylene glycol,
(f) about 14.8 to 46.7 wt % 1-(1,2,3,5,6,7,8,8a-octahydro-2,3,8,8-tetramethyl-2-naphthyl)ethan-1-one, or
(g) about 21.8 to 24.7 wt % kharismal.

12. A fragrance composition for imparting a liking attribute comprising
(a) about 0.05 to 2.0 wt % Amber Xtreme, and
at least one of the following:
(b) about 0.1 to 0.6 wt % geraniol,
(c) about 1.3 to 3.0 wt % helional,
(d) about 1.8 to 13.8 wt % muscemor,
(e) about 0.9 to 19.0 wt % ambrettolide.

13. A consumer product comprising the fragrance composition of claim 12.

14. The consumer product of claim 13, wherein said product is formulated for a consumer in North America, South America or Asia.

15. A method for imparting a liking attribute to a fragrance composition or consumer product comprising adding to the fragrance composition or consumer product:
(a) about 0.05 to 2.0 wt % Amber Xtreme, and
at least one of the following:
(b) about 0.1 to 0.6 wt % geraniol,
(c) about 1.3 to 3.0 wt % helional,
(d) about 1.8 to 13.8 wt % muscemor,
(e) about 0.9 to 19.0 wt % ambrettolide,
thereby imparting a liking attribute to the fragrance composition or consumer product.
